(19) United States
(12) Patent Application Publication
(10) Pub. No.: US 2025/0152776 A1
(43) Pub. Date: May 15, 2025

(54) CFR-PEEK ORTHOPEDIC IMPLANT AND PREPARATION METHOD THEREFOR, AND WIRELESS SENSING DEVICE

(71) Applicant: Tsinghua University, Beijing City (CN)

(72) Inventors: Xining Zang, Beijing City (CN); Zhe Zhao, Beijing City (CN); Jincai Huang, Beijing City (CN); Qing'ang Li, Beijing City (CN); Yiwei He, Beijing City (CN)

(21) Appl. No.: 18/722,720

(22) PCT Filed: Nov. 8, 2022
(86) PCT No.: PCT/CN2022/130564

§ 371 Date: Jun. 21, 2024

(30) Foreign Application Priority Data
Dec. 27, 2021 (CN) 202111618690.3

(51) Int. Cl. A61L 27/30; A61F 2/30

(52) U.S. Cl. CPC A61L 27/306; A61F 2/3094; A61L 27/303; A61F 2002/3097

(57) ABSTRACT

The present disclosure discloses a CFR-PEEK orthopedic implant and a preparation method therefor, and a wireless sensing device. The CFR-PEEK orthopedic implant includes a CFR-PEEK matrix and a patterned carbonization layer. The patterned carbonization layer is formed by performing in-situ carbonization of the CFR-PEEK matrix.

### TECHNICAL FIELD

The present disclosure relates to the field of wireless sensing, particularly to a CFR-PEEK orthopedic implant and a preparation method therefor, and a wireless sensing device.

### BACKGROUND

Monitoring of postoperative healing in orthopedics has always been an important and difficult research topic. In the process of postoperative rehabilitation, different weight bearing and activity patterns will affect a force suffered by and a slight motion of a fracture part, thereby having an irreversible influence on the healing of the part.

Currently, the most commonly used postoperative observation method is an X-ray plain film or X-Ray Computer Tomography (CT). However, the ionizing radiation of X-rays and the inconvenience of follow-up visits result in a low sampling frequency for this method, and fail to meet a demand on convenient monitoring at any time.

Therefore, if real-time detection and continuous monitoring of an orthopedic injury part can be performed, surgery and postoperative rehabilitation can be guided better, thereby a complication can be found and treated more timely.

### SUMMARY

The present disclosure is made based on the inventor's discovery and understanding of the following facts and issues.

As described above, an X-ray plain film or X-Ray CT cannot conveniently detect healing of an injured part at any time. A method of additionally adhering a sensor to an orthopedic implant and detecting the healing of an injured part by using an extracorporeal antenna to detect signals has obvious shortcomings: the additionally adhered sensor is adhered to a metal implant by a glue, after working in a body for a long time, the viscosity of the adhesion layer (i.e., the glue layer) decreases, which leads to a poor adherence of the sensor to the metal implant matrix, resulting in a stiffness mismatch problem, which consequently leads to a decrease in the capacitance C and the inductance L of the whole system. According to the resonant frequency calculation formula RRF=1/(2π\sqrt{L·C}), when the capacitance and the inductance decrease, the resonant frequency will increase, making the resonant response frequency of the sensor itself high, which consequently affects a difficulty of in-vitro detecting and the wireless-sensing-signal receiving, meanwhile, the sensitivity of the sensor will also be greatly reduced, which is not conducive to real-time detection and continuous monitoring of the injured part.

Carbon fiber reinforced polyether-ether-ketone (CFR-PEEK) has comprehensive performance such as excellent mechanical performance, chemical erosion resistance performance and biological compatibility, etc. Through a large number of experimental studies, the inventor found that a patterned carbonization layer can be formed by performing surface carbonization of the CFR-PEEK, the carbonization layer can be used to sense a mechanical change, temperature change or chemical change of an injured part in a body, and these changes can be transferred to an external terminal through signal conversion and transmission, so that healing of the injured part can be known via signals obtained from the terminal.

In view of this, it is one aspect of the present disclosure to provide a CFR-PEEK orthopedic implant, including a CFR-PEEK matrix and a patterned carbonization layer. The patterned carbonization layer is formed by performing in-situ carbonization of the CFR-PEEK matrix. Thus, the patterned carbonization layer formed in situ on the CFR-PEEK matrix may be used to sense a mechanical signal, temperature signal or chemical signal, and may be used in conjunction with an in-vitro probe or an in-vivo signal transduction device to detect and monitor the healing of an injured part in real time, so that a complication can be found in time, and surgery and postoperative rehabilitation can be guided better.

According to embodiments of the present disclosure, the CFR-PEEK orthopedic implant further includes a circuit structure which is provided near the patterned carbonization layer. The circuit structure includes a Wheatstone bridge circuit, an ADC module and a Bluetooth module. The circuit structure receives a resistance signal of the patterned carbonization layer, the resistance signal is converted into a voltage signal through the Wheatstone bridge circuit and is amplified to become an amplifying signal, and the amplifying signal is performed with analog-to-digital conversion through the ADC module and then is transmitted out through the Bluetooth module.

According to embodiments of the present disclosure, the patterned carbonization layer satisfies at least one of the following conditions: the patterned carbonization layer has a slender strip shape and is used to feed back a mechanical signal; the patterned carbonization layer is a curved connection structure with a plurality of slender lines and is used to feed back a temperature signal; and the patterned carbonization layer is square and is used to feed back a chemical signal. Thus, patterned carbonization layers having different shapes can be used to feed back different signals, which can reflect healing of an injured part more comprehensively.

It is another aspect of the present disclosure to provide a method for preparing a CFR-PEEK orthopedic implant, including: providing a CFR-PEEK matrix; and performing in-situ carbonization of a surface of the CFR-PEEK matrix, to form a patterned carbonization layer. Thus, the patterned carbonization layer may be formed by using in-situ carbonization, the CFR-PEEK orthopedic implant is used to treat fractures, etc., and the patterned carbonization layer formed by in-situ carbonization may be used to sense a mechanical signal, temperature signal, chemical signal of an injured part, which is conducive to real-time detection and monitoring of the injured part, timely detection of a complication, and better guidance for surgery and postoperative recovery.

According to embodiments of the present disclosure, the patterned carbonization layer satisfies at least one of the following conditions: the patterned carbonization layer has a slender strip shape and is used to feed back a mechanical signal; the patterned carbonization layer is a curved connection structure with a plurality of slender lines and is used to feed back a temperature signal; and the patterned carbonization layer is square and is used to feed back a chemical signal.

According to embodiments of the present disclosure, the in-situ carbonization is performed by laser irradiation.

According to embodiments of the present disclosure, the laser irradiation adopts ultraviolet nanosecond laser, and adopts a power of 5 W-10 W, a repetition frequency of 40 kHz-100 kHz, a scanning speed of 20-110 mm/s, and a defocusing amount of 2-10 mm.

It is still another aspect of the present disclosure to provide a wireless sensing device, including the aforementioned CFR-PEEK orthopedic implant, and the CFR-PEEK orthopedic implant is used to be implanted into a human body or an animal body. Thus, the wireless sensing device has all the features and advantages of the aforementioned CFR-PEEK orthopedic implant, which are not repeated here. In general, the wireless sensing device can detect in real time and continuously monitor the healing of the injured part, and can better guide surgery and postoperative healing.

According to embodiments of the present disclosure, the CFR-PEEK orthopedic implant further includes a circuit structure which is provided near the patterned carbonization layer. The circuit structure includes a Wheatstone bridge circuit, an ADC module and a Bluetooth module. The circuit structure receives a resistance signal of the patterned carbonization layer, the resistance signal is converted into a voltage signal through the Wheatstone bridge circuit and is amplified to become an amplifying signal, and the amplifying signal is performed with analog-to-digital conversion through the ADC module to be converted into a digital voltage signal, the digital voltage signal is then transmitted out through the Bluetooth module. The wireless sensing device further includes a mobile terminal which is used to receive a digital voltage signal transmitted by the Bluetooth module, and convert the digital voltage signal into the resistance signal and display it.

According to embodiments of the present disclosure, a wireless sensing device includes the aforementioned CFR-PEEK orthopedic implant, and the CFR-PEEK orthopedic implant is used to be implanted into a human body or an animal body. The wireless sensing device further includes an in-vitro probe and a network analyzer. The in-vitro probe releases a first alternating electromagnetic field, after the patterned carbonization layer senses a mechanical signal, a temperature signal or a chemical signal, a resonant frequency of the patterned carbonization layer will change, and the patterned carbonization layer generates an induced current under the first alternating electromagnetic field to generate a second alternating electromagnetic field, the second alternating electromagnetic field causes the first alternating electromagnetic field to change, the in-vitro probe senses a change in the first alternating electromagnetic field and transmits it to the network analyzer, and the network analyzer converts the change of the first alternating electromagnetic field into an alternating current signal and converts the alternating current signal into an offset of a peak value of the resonant frequency and displays the offset, so as to obtain information on the mechanical signal, chemical signal or temperature signal at an orthopedic implant injury in a human or an animal by analysis.

### BRIEF DESCRIPTION OF DRAWINGS

The above and/or additional aspects and advantages of the present disclosure will become obvious and easily understood from the description of embodiments in conjunction with the following drawings. In the drawings:

FIG. 1 shows a structural schematic diagram of a carbon fiber reinforced polyether-ether-ketone orthopedic implant according to an embodiment of the present disclosure;

FIG. 2 shows a structural schematic diagram of a carbon fiber reinforced polyether-ether-ketone orthopedic implant according to another embodiment of the present disclosure;

FIG. 3 shows a structural schematic diagram of a carbon fiber reinforced polyether-ether-ketone orthopedic implant according to still another embodiment of the present disclosure;

FIG. 4 shows a structural schematic diagram of a carbon fiber reinforced polyether-ether-ketone orthopedic implant according to yet still another embodiment of the present disclosure;

FIG. 5 shows a structural schematic diagram of a wireless sensing device according to an embodiment of the present disclosure;

FIG. 6 shows a structural schematic diagram of a wireless sensing device according to another embodiment of the present disclosure;

FIG. 7 shows a physical drawing of a network analyzer according to an embodiment of the present disclosure; and

FIG. 8 shows a three-point bending test result of a carbon fiber reinforced polyether-ether-ketone orthopedic implant according to an embodiment of the present disclosure.

### DESCRIPTION OF EMBODIMENTS

Embodiments of the present disclosure are described in detail below. It should be noted that the embodiments described below are exemplary and are intended only to explain the present disclosure and cannot be understood as limitations on the present disclosure. If a specific technology or condition is not indicated in an embodiment, technologies or conditions described in the literatures in the art, or product specifications shall be followed. A reagent or an instrument used, in which a manufacturer is not indicated, are conventional products that can be obtained through market purchase.

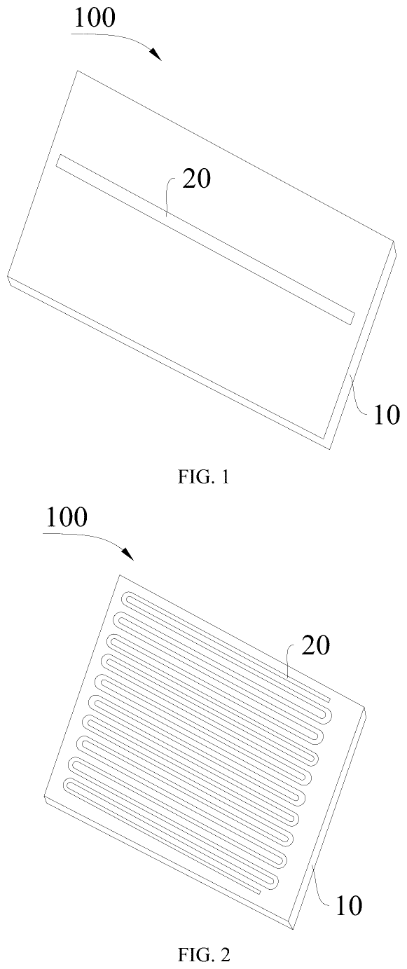

It is one aspect of the present disclosure to provide a carbon fiber reinforced polyether-ether-ketone (CFR-PEEK) orthopedic implant, referring to FIGS. 1 to 3, the CFR-PEEK orthopedic implant 100 includes a CFR-PEEK matrix 10 and a patterned carbonization layer 20. The patterned carbonization layer 20 is formed by performing in-situ carbonization of the CFR-PEEK matrix 10. Thus, the patterned carbonization layer formed in situ on the CFR-PEEK matrix can be used to sense a mechanical signal, temperature signal or chemical signal in a body, and can be used in conjunction with an in-vitro probe or an in-vivo signal transduction device (such as a circuit structure, etc.) to detect and monitor the healing of an injured part in real time, so that a complication can be found in time, and surgery and postoperative rehabilitation can be guided better. In addition, since the patterned carbonization layer is formed on the matrix through in-situ carbonization, the bonding between the patterned carbonization layer and the matrix is very firm, without the problem of weak bonding as the time passes, and thus without the problem of a significant increase in the resonance response frequency.

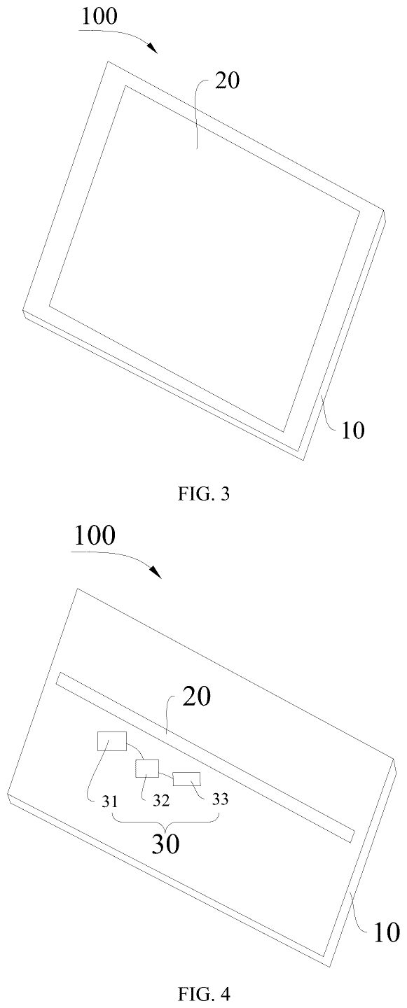

According to the embodiments of the present disclosure, the patterned carbonization layer 20 in the CFR-PEEK orthopedic implant 100 may have different patterns to feed back different signal features. Specifically, according to a specific embodiment of the present disclosure, referring to FIG. 1, the patterned carbonization layer 20 may be in a slender strip shape. The patterned carbonization layer having a slender strip shape is more sensitive to a mechanical signal, and can accurately and timely respond to the mechanical signal, so that the patterned carbonization layer 20 may be used to feed back a mechanical signal, such as a stress, a strain, etc. According to another specific embodiment of the present disclosure, referring to FIG. 2, the patterned carbonization layer 20 may be a curved connection structure with a plurality of slender lines, this structure can sense a subtle change in temperature, so that the patterned carbonization layer 20 may be used to feed back a temperature signal. According to still another specific embodiment of the present disclosure, referring to FIG. 3, the patterned carbonization layer 20 may be square, this square patterned carbonization layer can sense a change in a liquid by absorbing compositions of the liquid, so that the patterned carbonization layer 20 can be used to feed back a chemical signal, such as a change in pH in a body, etc. It should be noted that the above shapes of the patterned carbonization layer in the present disclosure are only for explanation rather than for limiting the present disclosure.

According to embodiments of the present disclosure, referring to FIG. 4, the CFR-PEEK orthopedic implant 100 may further include a circuit structure 30 which is provided near the patterned carbonization layer 20. The circuit structure includes a Wheatstone bridge circuit 31, an ADC (analog-digital conversion) module 32, and a Bluetooth module 33. The patterned carbonization layer 20 senses different signals and transmits them to the circuit structure 20 in the form of a resistance signal. The circuit structure 20 can receive the resistance signal of the patterned carbonization layer 20, and the resistance signal is converted to a voltage signal through the Wheatstone bridge circuit 31 and is amplified to become an amplifying signal. Since the amplifying signal is a virtual voltage signal and cannot be transmitted through the Bluetooth module, the amplifying signal needs to be performed with analog-digital conversion through the ADC module 32 to be converted into a digital voltage signal, and then is transmitted out through the Bluetooth module 33.

It is another aspect of the present disclosure to provide a method for preparing a CFR-PEEK orthopedic implant, including:

S100: providing a CFR-PEEK matrix.

In this step, a CFR-PEEK matrix is provided. The CFR-PEEK is a polyether-ether-ketone composite material with a reinforced carbon fiber, concentrates advantages of a polyether-ether-ketone material and a carbon fiber material, has a light mass, and has excellent mechanical performance, excellent chemical erosion resistance performance and excellent biological compatibility, so that the CFR-PEEK can be used as an orthopedic implant.

S200: performing in-situ carbonization of a surface of the CFR-PEEK matrix.

In this step, a patterned carbonization layer is formed by performing in-situ carbonization of a surface of the CFR-PEEK matrix, so by this simple method, the patterned carbonization layer can be formed on a surface of the CFR-PEEK matrix. The pattered carbonization layer in the CFR-PEEK orthopedic implant can be used to sense a mechanical signal, temperature signal or chemical signal in a body, which is conducive to real-time detection and real-time monitoring of healing of an injured part.

The shapes of the patterned carbonization layer have been described in detail in the preceding text, and are not repeated here.

According to the embodiments of the present disclosure, a specific mode of performing in-situ carbonization of the CFR-PEEK matrix may be laser irradiation. Specifically, laser may be directly irradiated on a surface of the CFR-PEEK, so that a carbonization conductive layer with high conductivity is carbonized on an accurately selected region of the surface of the CFR-PEEK by means of instantaneous high-temperature laser carbonization. The carbonization conductive layer is directly used as an in-situ sensor for sensing a mechanical signal, temperature signal or chemical signal in a body.

According to the embodiments of the present disclosure, laser irradiation may use ultraviolet light, visible light, or infrared light, and may use a pulse width of millisecond, nanosecond, picosecond, or femtosecond, etc., as long as it is capable of carbonizing a required patterned carbonization layer on the surface of the CFR-PEEK. In addition, an energy density of the laser may be changed by changing laser parameters such as an output power, a scanning speed, a repetition rate, defocusing amount of laser, thereby changing the morphology, compositions and resistivity of the patterned carbonization layer. Meanwhile, it is also possible to design a pattern of the carbonization conductive layer (carbonization layer) by designing a laser processing trajectory, to form a sensor having different patterns and applicable for composite signal sensing such as mechanics (pressure, strain, friction, etc.), temperature, chemistry (pH, etc.).

According to some embodiments of the present disclosure, ultraviolet nanosecond laser may be adopted for laser irradiation, an output power of a laser may be 5 W-10 W, for example may be 5 W, 6 W, 7 W, 8 W, 9 W, 10 W, etc., a repetition frequency may be 40 kHz-100 kHz, for example may be 40 kHz, 50 kHz, 60 kHz, 70 kHz, 80 kHz, 90 kHz, 100 kHz, etc., and a scanning speed may be 20-110 mm/s, for example may be 20 mm/s, 30 mm/s, 40 mm/s, 50 mm/s, 60 mm/s, 70 mm/s, 80 mm/s, 90 mm/s, 100 mm/s, 110 mm/s, etc., and a defocusing amount may be 2-10 mm, for example may be 2 mm, 3 mm, 4 mm, 5 mm, 6 mm, 7 mm, 8 mm, 9 mm, 10 mm, etc. Thus, an energy density of irradiating on the surface of the CFR-PEEK matrix may be greater than 0.83 J/mm², thereby forming the carbonization layer on the surface of the CFR-PEEK matrix.

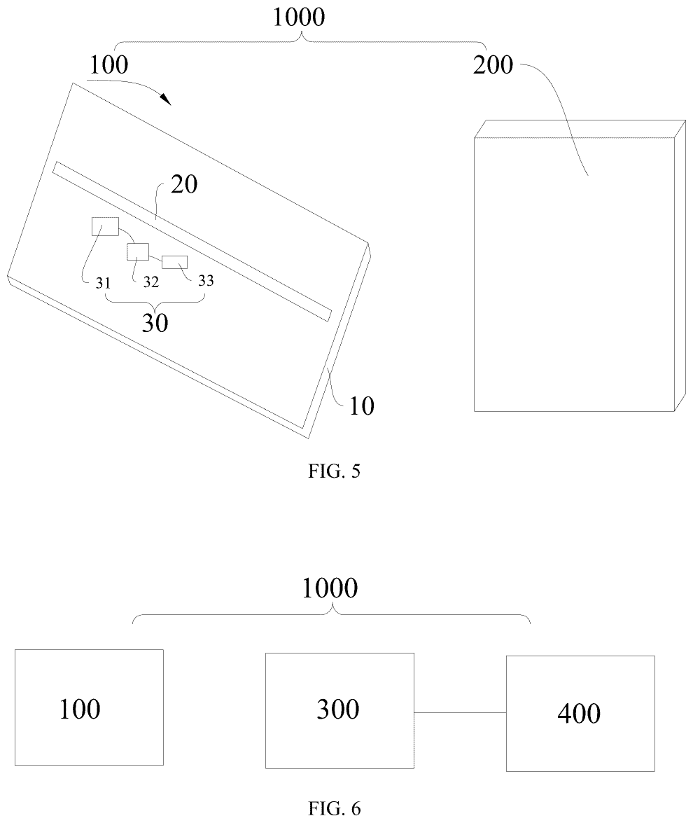

It is still another aspect of the present disclosure to provide a wireless sensing device, referring to FIG. 5 and FIG. 6, the wireless sensing device 1000 includes the aforementioned CFR-PEEK orthopedic implant 100 that is used to be implanted into a human body or an animal body. The wireless sensing device has all the features and advantages of the aforementioned CFR-PEEK orthopedic implant, which are not repeated here. In general, the wireless sensing device may be used for real-time detection and real-time monitoring of the healing of an injured part, can timely feed back a mechanical signal, temperature signal and chemical signal of the injured part, which is convenient for follow-up, and can be used to guide surgery and postoperative rehabilitation treatment.

According to some embodiments of the present disclosure, referring to FIG. 5, the CFR-PEEK orthopedic implant 100 further includes a circuit structure 30 which is provided near the patterned carbonization layer 20. The circuit structure 30 includes a Wheatstone bridge circuit 31, an ADC module 32, and a Bluetooth module 33. The circuit structure 30 receives a resistance signal of the patterned carbonization layer 20, the resistance signal is converted into a voltage signal through the Wheatstone bridge circuit 31 and is amplified to become an amplifying signal, and the amplifying signal is performed with analog-to-digital conversion through the ADC module 32 to be converted into a digital voltage signal, the digital voltage signal is then transmitted out through the Bluetooth module 32. Referring to FIG. 5, the wireless sensing device 1000 further includes a mobile terminal 200 which is used to receive a digital voltage signal transmitted by the Bluetooth module 33, and convert the digital voltage signal into a resistance signal and display the resistance signal. It should be noted that the mobile terminal may be a device or apparatus with a display function, such as a mobile phone or a computer, etc.

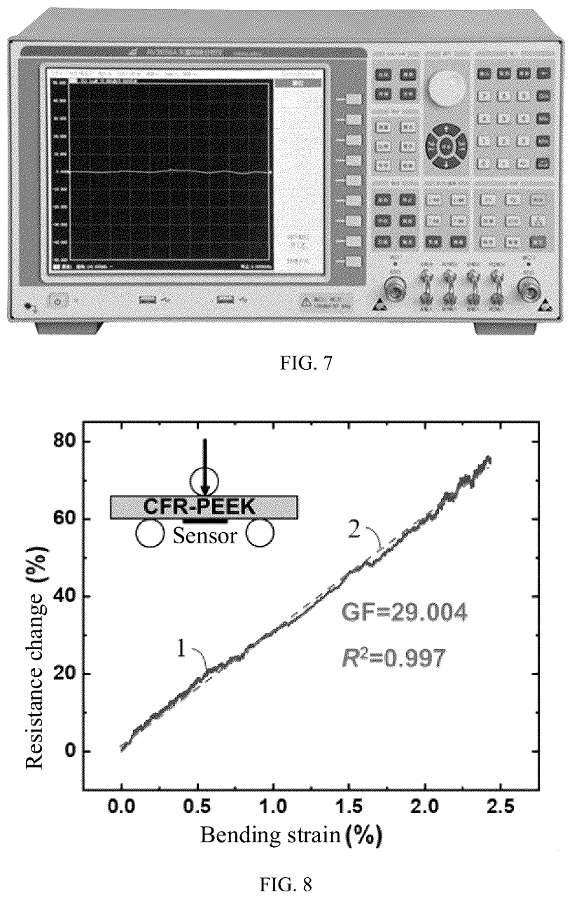

According to some embodiments of the present disclosure, referring to FIG. 6, the wireless sensing device 1000 comprises a CFR-PEEK orthopedic implant 100, and further includes an in-vitro probe 300 and a network analyzer 400. FIG. 7 is a physical drawing of the network analyzer 400, which includes a display screen and a plurality of adjustment buttons, etc. The working principle of the wireless sensing device 1000 is provided as follows:

The in-vitro probe 300 releases a first alternating electromagnetic field; after the patterned carbonization layer 20 senses a mechanical signal, a temperature signal or a chemical signal, a resonant frequency of the patterned carbonization layer 20 will change, and the patterned carbonization layer 20 generates an induced current under the first alternating electromagnetic field to generate a second alternating electromagnetic field, the second alternating electromagnetic field will cause the first alternating electromagnetic field to change; the in-vitro probe 300 senses a change in the first alternating electromagnetic field and transmits it to the network analyzer 400; the network analyzer 400 converts the change of the first alternating electromagnetic field into an alternating current signal, and converts the alternating current signal into an offset of a peak value of the resonant frequency and displays the offset, so as to obtain change information on the mechanical signal, chemical signal or temperature signal at an orthopedic implant injury in a human or an animal by analysis.

The solution of the present disclosure is described via the following specific embodiment.

### Embodiment 1

Ultraviolet nanosecond laser is used to irradiate a surface of a CFR-PEEK (CFR-PEEK) matrix, the ultraviolet nanosecond laser used has a wavelength of 355 nm and a pulse width of 25 ns, and a laser used has an output power of 5.5 W, a repetition rate of 40 kHz, a scanning speed of 60 mm/s, and a defocusing amount of 2 mm, so that a patterned carbonization layer is formed on the surface of the CFR-PEEK matrix to obtain a CFR-PEEK orthopedic implant.

A three-point bending text is carried out on the CFR-PEEK orthopedic implant, and the text result is shown FIG. 8, in which the ordinate resistance change refers to a ratio of an absolute value of a resistance change to an initial resistance value, expressed as a percentage, the initial resistance value refers to a resistance value measured when no bending strain occurs, and the absolute value of the resistance change refers to an absolute value of a difference value between a resistance value measured after bending strain occurs and the initial resistance value. The curve 1 is a curve in which a resistance change obtained by an actual test varies with bending strain, and the dashed line 2 is obtained by origin software fitting. Upon calculation, within a working strain range of 0-2.5%, the orthopedic implant has a higher degree of linearity (R²=0.997), the degree of linearity is close to 1, indicating that use of this orthopedic implant to sense a signal change has good reliability and the repeatability is higher. Meanwhile, the test shows that this orthopedic implant also has higher sensitivity (Gauge Factor (GF), the sensitivity is 29.0074, the sensitivity is higher, indicating that this orthopedic implant is more sensitive to a signal change and can be used to detect a tiny signal change.

Through the above experiment, it can be known that the patterned carbonization layer in the CFR-PEEK orthopedic implant can better feed back a mechanical signal, this orthopedic implant can be used in a human or animal body, and can be used in conjunction with an in-vitro probe or an in-vivo peripheral circuit to achieve real-time detection and real-time monitoring of the healing of an injured part.

In the present disclosure, the healing of the injury refers to healing of an injury at a fracture part. During a gradual healing process of the injured part, the bending strain sensed by the patterned carbonization layer will gradually decrease. Under normal circumstances, when the healing is successful, the bending strain sensed by the patterned carbonization layer will be about 2%, at this moment, a corresponding resistance change in FIG. 8 is about 60%. That is, if the resistance change sensed by the patterned carbonization layer is greater and gradually decreases as time passes, it can be considered that the injured part is gradually healing; however, if the resistance change suddenly increases, it can be considered that inflammation or other complications occur in the injured part, corresponding treatment measures may be taken in time; and if the resistance change of the patterned carbonization layer is about 60% or less, it can be considered that the injured part is healed successfully. For a temperature signal, if a temperature sensed by the patterned carbonization layer is slightly higher than 37° C., it can be considered that the injured part has not completely healed, while if the temperature sensed by the patterned carbonization layer is 37° C. or slightly lower than 37° C., it can be considered that the injured part has successfully healed.

In the description of the present Specification, description of reference terms “one embodiment”, “another embodiment”, “a further embodiment”, “a specific embodiment”, “another specific embodiment”, “a further specific embodiment” and “some embodiments”, etc. means that a specific feature, structure, material or characteristic described in conjunction with this example is included in at least one example of the present disclosure. In the present Specification, schematic representations of the above terms are not necessary to aim at the same examples. And, the described specific feature, structure, material or characteristic may be combined in a suitable manner in any one or more examples. In addition, without contradicting each other, persons skilled in the art may incorporate and combine different examples described in the present Specification as well as the features of different examples. In addition, it should be noted that in the present Specification, the terms “first” and “second” are used for the descriptive purpose only and cannot be understood as indicating or implying relative importance or as implicitly indicating the number of the indicated technical features.

Although examples of the present disclosure have been shown and described above, it can be understood that the above examples cannot be understood as limitations on the present disclosure, persons skilled in the art may change, amend, replace or modify the above examples within the scope of the present disclosure.

1. A CFR-PEEK orthopedic implant, comprising a CFR-PEEK matrix and a patterned carbonization layer;
wherein the patterned carbonization layer is formed by performing in-situ carbonization of the CFR-PEEK matrix.

2. The CFR-PEEK orthopedic implant according to claim 1, further comprising a circuit structure which is provided near the patterned carbonization layer, and the circuit structure comprises a Wheatstone bridge circuit, an ADC module, and a Bluetooth module; and
the circuit structure receives a resistance signal of the patterned carbonization layer, the resistance signal is converted into a voltage signal through the Wheatstone bridge circuit and is amplified to become an amplifying signal, and the amplifying signal is performed with analog-to-digital conversion through the ADC module and then is transmitted out through the Bluetooth module.

3. The CFR-PEEK orthopedic implant according to claim 1, the patterned carbonization layer satisfies at least one of the following conditions:
the patterned carbonization layer has a slender strip shape and is used to feed back a mechanical signal;
the patterned carbonization layer is a curved connection structure with a plurality of slender lines and is used to feed back a temperature signal; and
the patterned carbonization layer is square and is used to feed back a chemical signal.

4. A method for preparing a CFR-PEEK orthopedic implant, comprising:
providing a CFR-PEEK matrix; and
performing in-situ carbonization of a surface of the CFR-PEEK matrix, to form a patterned carbonization layer.

5. The method according to claim 4, wherein the patterned carbonization layer satisfies at least one of the following conditions:
the patterned carbonization layer has a slender strip shape and is used to feed back a mechanical signal;
the patterned carbonization layer is a curved connection structure with a plurality of slender lines and is used to feed back a temperature signal; and
the patterned carbonization layer is square and is used to feed back a chemical signal.

6. The method according to claim 4 or 5, wherein the in-situ carbonization is performed by laser irradiation.

7. The method according to claim 6, wherein the laser irradiation adopts ultraviolet nanosecond laser, and adopts a power of 5 W-10 W, a repetition frequency of 40 kHz-100 kHz, a scanning speed of 20-110 mm/s, and a defocusing amount of 2-10 mm.

8. A wireless sensing device, comprising a CFR-PEEK orthopedic implant, the CFR-PEEK orthopedic implant being used to be implanted into a human body or an animal body, wherein the CFR-PEEK orthopedic implant comprises a CFR-PEEK matrix and a patterned carbonization layer, and the patterned carbonization layer is formed by performing in-situ carbonization of the CFR-PEEK matrix.

9. The wireless sensing device according to claim 8, wherein the CFR-PEEK orthopedic implant further includes a circuit structure provided near the patterned carbonization layer;
wherein the circuit structure comprises a Wheatstone bridge circuit, an ADC module and a Bluetooth module;
the circuit structure receives a resistance signal of the patterned carbonization layer, the resistance signal is converted into a voltage signal through the Wheatstone bridge circuit and is amplified to become an amplifying signal, the amplifying signal is performed with analog-to-digital conversion through the ADC module to be converted into a digital voltage signal, and the digital voltage signal is then transmitted out through the Bluetooth module; and
the wireless sensing device further comprises a mobile terminal which is used to receive a digital voltage signal transmitted by the Bluetooth module, and convert the digital voltage signal into the resistance signal and display it.

10. The wireless sensing device according to claim 8, wherein the wireless sensing device further comprises an in-vitro probe and a network analyzer; and
the in-vitro probe releases a first alternating electromagnetic field, after the patterned carbonization layer senses a mechanical signal, a temperature signal or a chemical signal, a resonant frequency of the patterned carbonization layer will change, and the patterned carbonization layer generates an induced current under the first alternating electromagnetic field to generate a second alternating electromagnetic field, the second alternating electromagnetic field causes the first alternating electromagnetic field to change, the in-vitro probe senses a change in the first alternating electromagnetic field and transmits it to the network analyzer, the network analyzer converts the change of the first alternating electromagnetic field into an alternating current signal and converts the alternating current signal into an offset of a peak value of the resonant frequency and displays the offset, so as to obtain change information on the mechanical signal, chemical signal or temperature signal at an orthopedic implant injury in a human or an animal by analysis.

11. The wireless sensing device according to claim 8, wherein the CFR-PEEK orthopedic implant further comprises a circuit structure which is provided near the patterned carbonization layer, and the circuit structure comprises a Wheatstone bridge circuit, an ADC module, and a Bluetooth module; and
the circuit structure receives a resistance signal of the patterned carbonization layer, the resistance signal is converted into a voltage signal through the Wheatstone bridge circuit and is amplified to become an amplifying signal, and the amplifying signal is performed with analog-to-digital conversion through the ADC module and then is transmitted out through the Bluetooth module.

12. The wireless sensing device according to claim 8, wherein the patterned carbonization layer satisfies at least one of the following conditions:
the patterned carbonization layer has a slender strip shape and is used to feed back a mechanical signal;
the patterned carbonization layer is a curved connection structure with a plurality of slender lines and is used to feed back a temperature signal; and
the patterned carbonization layer is square and is used to feed back a chemical signal.
